(19) United States
(12) Patent Application Publication
(10) Pub. No.: US 2002/0115716 A1
(43) Pub. Date: Aug. 22, 2002

(54) Oral low dose butyrate compositions

(76) Inventors: Pravin Chaturvedi, Andover, MA (US); Michael Su, Newton, MA (US); Roger Tung, San Diego, CA (US)

Correspondence Address: VERTEX PHARMACEUTICALS INCORPORATED, 130Waverly Street, Cambridge, MA, 02130-4646, US

(21) Appl. No.: 09/955,707

(22) Filed: Sep. 19, 2001

Related U.S. Application Data
(60) Provisional application No. 60/125,607, filed on Mar. 19, 1999.

(51) Int. Cl. A61K 31/215; A61K 31/19

(52) U.S. Cl. 514/529; 514/547; 514/570

(57) ABSTRACT

This invention relates to orally available compositions which deliver an amount of butyrate or a butyrate analogue effective to ameliorate β-hemoglobinopathies, such as β-thalassemia and sickle cell anemia, cystic fibrosis, cancer and other diseases which are known to be treatable with butyrate. The invention also relates to methods of treating these diseases with such low dose oral compositions.

### CROSS-REFERENCE TO RELATED APPLICATIONS

This application claims priority to co-pending International Patent Application PCT/US00/07128, filed Mar. 17, 2000, which claims priority of United States provisional application Ser. No. 60/125,607, which was filed Mar. 19, 1999. The entirety of which is herein incorporated by reference.

### TECHNICAL FIELD OF THE INVENTION

This invention relates to orally available compositions which deliver an amount of butyrate or a butyrate analogue effective to ameliorate β-hemoglobinopathies, such as β-thalassemia and sickle cell anemia, cystic fibrosis, cancer and other diseases which are known to be treatable with butyrate. The invention also relates to methods of treating these diseases with such low dose oral compositions.

### BACKGROUND OF THE INVENTION

Recent studies have suggested that butyrate or analogues thereof are useful in treating a wide variety of diseases. For example, butyrate has been implicated in increasing fetal hemoglobin (HbF) levels, which in turn, can ameliorate the effects of β-hemoglobinopathies, such as sickle cell anemia and β-thalassemia [S. Perrine et al., A Short Term Trial of Butyrate to Stimulate Fetal-Globin-Gene Expression in the β-globin Disordersμ, N. Eng. J. Med., 328, pp. 81-86 (1993); S. P. Perrine et. al., “Isobutyramide, an Orally Bioavailable Butyrate Analogue, Stimulates Fetal Globin Gene Expression In Vitro and In Vivo”, British J. Haematology, 88, pp. 555-61 (1994); A. F. Collins et al., “Oral Sodium Phenylbutyrate Therapy in Homozygous β Thalassemia: A Clinical Trial”, Blood, 85, pp. 43-49 (1995); see also U.S. Pat. Nos. 4,822,821, Re 36,080, and PCT publication WO097/12855].

Butyrate has also been shown to induce cell differentiation [A. Leder and P. Leder, “Butyric Acid, a Potent Inducer of Erythroid Differentiation in Cultured Erythroleukemic Cells”, Cell, 5, pp. 319-22 (1975)]. This led to studies examining the effects of butyrate, either alone or in combination with other drugs, on various cancers, such as leukemia and tumors [A. Novogrodsky et al., “Effect of Polar Organic Compounds on Leukemic Cells”, Cancer, 51, pp. 9-14 (1983); C. Chany and I. Cerutti, “Antitumor Effect Of Arginine Butyrate in Conjunction with Corynebacterium Parvum and Interferon”, Int. J. Cancer, 30, pp. 489-93 (1982); M. Otaka et al., “Antibody-Mediated Targeting of Differentiation Inducers To Tumor Cells: Inhibition of Colonic Cancer Cell Growth in vitro and in vivo”, Biochem. Biophys. Res. Commun., 158, pp. 202-08 (1989); O. Vincent-Fiquet et al., “Effects of Arginine Butyrate and Tributyrylxylitol on Cultured Human Sarcoma Cells”, Anticancer Research, 14, pp. 1823-28 (1994)]; pancreatic cancer [S. Corra et al., “Modification of Antigen Expression in Human and Hamster Pancreatic Cancer Cell Lines Induced by Sodium Butyrate”, Teratogenesis, Carcinogenesis, and Mutagenesis, 13, pp. 199-215 (1993)]; colon cancer [Y. Tanaka et al. “Enhancement of Butyrate Induced Differentiation of HT-29 Human Colon Carcinoma Cells by 1,25-Dihydroxyvitamin D₃”, Biochem. Pharmacol. 38, pp. 3859 (1989); P. Perrin et al., “An Interleukin 2/Sodium Butyrate Combination as Immunotherapy for Rat Colon Cancer Peritoneal Carcinomatosis”, O. C. Velazquez et al., “Implications for Neoplasia”, Dig. Dis. Sci., 41, pp. 727-39 (1996); A. Hague et al., “Apoptosis in Colorectal Tumour Cells: Induction by the Short Chain Fatty Acids Butyrate, Propionate And Acetate and by the Bile Salt Deoxycholate”, Int. J. Cancer, 60, pp. 400-6 (1995); J. Dang et al., “Sodium Butyrate Inhibits Expression Of Urokinase And Its Receptor mRNAs At Both Transcription And Posttranscription Levels In Colon Cancer Cells”, FEBS Letts., 359, pp. 147-50 (1995); and J. A. McBain et al, “Phorbol Ester Augments Butyrate-Induced Apoptosis Of Colon Cancer Cells”, Int. J. Cancer, 67, pp. 715-723 (1996)]; and other cancers [P. R. Pouillart, Life Sciences, 63, pp. 1739-60 (1998); see also, PCT publications WO 96/15660 and WO 98/40064 and U.S. Pat. No. 5,763,488].

Butyrates have also been investigated for the treatment of inflammatory bowel diseases, such as colitis and Crohn's disease [W. Frankel et al., “Butyrate Increases Colonocyte Protein Synthesis In Ulcerative Colitis”, Journal of Surgical Research, 57, pp. 210-214 (1994); A. Finnie et al, “Colonic Mucin Synthesis is Increased by Sodium Butyrate”, Gut, 36, pp. 9-99 (1995); and PCT publication WO 98/40064].

More recently, it has been suggested that butyrate may be beneficial in the treatment of cystic fibrosis (CF) by properly directing the mutant, but functional gene product of the CFTR gene to the plasma membrane [S. H. Cheng et al., Am. J. Physiol., 268, pp. L615-L624 (1995); U.S. Pat. No. 5,750,571]. In connection with gene therapy, it has been shown that retroviral expression of the wild-type CFTR gene is enhanced in the presence of butyrate [J. C. Olsen et al., Hum. Gene Ther., 6, pp. 1195-1202 (1995)].

Because butyrate is well tolerated in mammals, trials of these drugs focused on administering high doses of butyrate or butyrate derivatives (or salts or prodrugs thereof) in the range of 250-2,000 mg/kg/day for extended periods of time. We postulate that such high dosages and long-term treatment are not necessary to produce the desired effect in oral forms of butyrate salts, prodrug and derivatives.

### SUMMARY OF THE INVENTION

The present invention solves these problems by providing compositions comprising 0.5 to 10 grams of an orally available butyrate prodrug, salt or analogue and a pharmaceutically acceptable carrier. These low dose compositions, which are designed to be administered from 1 to 4 times per day produce a serum butyrate blood concentration of between 10 and 200 μM for a period of between 1 to 8 hours.

The invention also provides methods of treating diseases responsive to butyrate and its analogues. These methods comprise administering to a patient an amount of an orally available butyrate prodrug, salt or analogue sufficient to produce and maintain a serum butyrate blood concentration of between 10 and 200 μM for a period of between 4 to 8 hours.

### DETAILED DESCRIPTION OF THE INVENTION

According to one embodiment, the present invention provides an orally available composition comprising:

a) an amount of a prodrug, salt or analogue of butyrate sufficient to maintain a serum butyrate concentration of between 10 and 200 μM for a period of 1 to 8 consecutive hours; and

b) a pharmaceutically acceptable carrier.

Prodrugs, salts and analogues of butyrate have been described in WO 96/15660 and in U.S. Pat. No. 5,763,488, the disclosures of which are herein incorporated by reference. Other orally available prodrugs, salts and analogues of butyrate that may be useful in this invention include, but are not limited to, tributyrine, ethylbutyryl lactate, pivalyloxymethyl butyrate (AN-9), AN-10 [A. Nudelman et al., “Novel Anticancer Prodrug of Butyric Acid”, J. Med. Chem., 35, pp. 687-94 (1992)], isobutyramide, 1-octyl butyrate, orthonitrobenzyl butyrate, monobutyrate-3-monoacetone glucose, monobutyrate-1-monoacetone mannose and monobutyrate xylitol, isobutyramide, 4-phenylbutyrate, and 4-phenyl acetate. Each of these compounds releases butyrate or a butyrate analogue into the blood stream upon administration.

The amount of the butyrate salt, prodrug or analogue necessary to produce the stated 10 to 200 μM blood level for 1 to 8 hours will depend upon how many moles of butyrate are released by the administered compound. For those compounds which release 1 mole of butyrate or butyrate analogue per mole of compound, this amount is between 0.5 to 10 grams. It should be noted that although tributyrin contains three butyrate moieties, only one is released from the molecule. The resulting dibutyrin molecule is excreted without further release of butyrate.

According to a preferred embodiment, the butyrate salt, prodrug or analogue used in the compositions of this invention is selected from ethylbutyryl lactate, tributyrin, 4-phenyl butyrate, AN-9 or AN-10.

The pharmaceutical compositions of this invention may be administered in any orally acceptable dosage form including, but not limited to, capsules, tablets, aqueous suspensions or solutions. In the case of tablets for oral use, pharmaceutically acceptable carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

The compositions of this invention are useful for treating a disease selected from a β-hemoglobinopathy, diseases characterized by neoplastic, tumorigenic or malignant cell growth (“malignant disease”), inflammatory bowel disease, or cystic fibrosis, for counteracting chemotherapy-induced mucocutaneous side effects or for enhancing the efficiency of gene therapy in a patient. Thus, the compositions of this invention may additionally comprise an agent that is normally used to treat the specific disorder that the butyrate composition will be used for.

In the case of β-hemoglobinopathies, the composition may additionally comprise hydroxyurea, clotrimazole, erythropoietin and salts of short-chain fatty acids, such as valproic acid.

17 For cancer treatment, the composition of this invention may additionally comprise erythropoietin, or a cancer chemotherapeutic agent, such as hydroxyurea or 5-azacytidine or 3-thiacytidine.

According to another embodiment, the invention provides methods for treating a disease selected from a β-hemoglobinopathy, diseases characterized by neoplastic, tumorigenic or malignant cell growth, malignant hematological disorders, inflammatory bowel disease, or cystic fibrosis, counteracting chemotherapy-induced mucocutaneous side effects or enhancing the efficiency of gene therapy in a patient. Each of these methods comprises the steps of:

a) treating the patient each day for 2 to 6 consecutive days with an amount of a prodrug, salt or analogue of butyrate sufficient to maintain a serum butyrate concentration of between 10 and 200 μM for a period of 4 to 8 consecutive hours; and

b) halting said treatment for a period of 15 to 30 consecutive days before reinitiating said treatment.

The treatment set forth above may be achieved with between 1 to 4 doses of the butyrate compound per day. The amount of butyrate compound necessary to achieve and maintain serum butyrate levels of between 10 to 200 μM are set forth above.

Examples of β-hemoglobinopathies that may be treated by the methods of this invention include, but are not limited to, sickle cell syndromes, such as sickle cell anemia, hemoglobin SC disease, hemoglobin SS disease and sickle β-thalassemia; β-thalassemia syndromes, such as β-thalassemia; other genetic mutations of the β-globin gene locus that lead to unstable hemoglobins, such as congenital Heinz body anemia, β-globin mutants with abnormal oxygen affinity and structural mutants of β-globin that result in thalassemic phenotype. These diseases are described in The Molecular Basis of Blood Disease, vol. II, G. Stamatoyannopoulos et at., eds., pp. 157-244 (1994).

Examples of malignant diseases that may be treated by the methods of this invention include, but are not limited to carcinomas, malignant hematological disorders, myelomas, melanomas, lymphomas and leukemias. More specifically, these diseases include colo-rectal cancer, lung cancer or prostate cancer. Treatment includes prevention of progression of the disease or its recurrence.

An example of a chemotherapy-induced mucocutaneous side effects that can be treated by the methods of this invention is alopecia.

Examples of inflammatory bowel diseases that may be treated by the methods of this invention include, but are not limited to colitis, pouchitis and Crohn's disease.

According to a preferred embodiment, the above-described method comprises the additional step of treating the patient with an agent that is normally used to treat such diseases. These agents are well known in the art and many are set forth above in the description of the compositions of this invention. The additional agent may be administered prior to, sequentially with (as a part of a single or a multiple dosage form) or after treatment with the butyrate compound.

The amount of conventional agent administered in these methods is preferably less than that normally required to treat such diseases in a monotherapy. The normal dosages of these conventional agents are well known in the art.

Combination therapies with conventional agents according to this invention (whether part of a single composition or administered separate from the butyrate prodrug, salt or analogue) may also exert an additive or synergistic effect, particularly when each component acts to treat or prevent the target disease via a different mechanism.

We claim:

1. An orally available composition comprising:
a) an amount of a prodrug, salt or analogue of butyrate sufficient to maintain a serum butyrate concentration of between 10 and 200 μM for a period of 1 to 8 consecutive hours; and
b) a pharmaceutically acceptable carrier.

2. The composition according to claim 1, wherein the amount of said prodrug, salt or analogue of butyrate is between 0.5 to 10 grams.

3. The composition according to claim 1 or 2, wherein said prodrug, salt or analogue of butyrate is selected from ethylbutyryl lactate, tributyrin, 4-phenyl butyrate, phenyl acetate, AN-9 or AN-10.

4. The composition according to claim 1 or 2, additionally comprising a conventional agent for treating a β-hemoglobinopathy in a patient.

5. The composition according to claim 1 or 2, additionally comprising a conventional agent for treating a malignant disease in a patient.

6. A method of treating a patient suffering from a disease selected from a β-hemoglobinopathy, a malignant disease, inflammatory bowel disease, or cystic fibrosis; counteracting chemotherapy-induced mucocutaneous side effects in patient; or enhancing the efficiency of gene therapy in a patient, comprising the steps of:
a) treating the patient each day for 2 to 6 consecutive days with an orally available amount of a prodrug, salt or analogue of butyrate sufficient to maintain a serum butyrate concentration of between 10 and 200 μM for a period of 4 to 8 consecutive hours; and
b) halting said treatment for a period of 15 to 30 consecutive days before reinitiating said treatment.

7. The method according to claim 6, wherein said patient is administered between 0.5 and 10 grams per day of said prodrug, salt or analogue of butyrate.

8. The method according to claim 7, wherein said prodrug, salt or analogue of butyrate is administered in 1 to 4 separate dosages per day.

9. The method according to claim 6, wherein said prodrug, salt or analogue of butyrate is selected from ethylbutyryl lactate, tributyrin, 4-phenyl butyrate, phenyl acetate, AN-9 or AN-10.

10. The method according to any one of claims 6 to 9, wherein said method is used to treat a β-hemoglobinopathy and wherein said method comprises the additional step of administering to said patient as part of a single of multiple dosage form a conventional agent for treating a β-hemoglobinopathy.

11. The method according to any one of claims 6 to 9, wherein said method is used to treat a malignant disease and wherein said method comprises the additional step of administering to said patient as part of a single of multiple dosage form a conventional agent for treating a malignant disease.
